(19) United States
(12) Patent Application Publication
(10) Pub. No.: US 2023/0329810 A1
(43) Pub. Date: Oct. 19, 2023

(54) SYSTEM AND METHOD FOR IMPLEMENTING A MULTI-TURN ROTARY CONCEPT IN AN ACTUATOR MECHANISM OF A SURGICAL ROBOTIC ARM

(71) Applicant: Vicarious Surgical Inc., Waltham, MA (US)

(72) Inventors: Matthew Kincaid, Medford, MA (US); Ryan Fish, Playa Vista, CA (US)

(21) Appl. No.: 18/213,011

(22) Filed: Jun. 22, 2023

Related U.S. Application Data
(63) Continuation of application No. PCT/US2021/064974, filed on Dec. 22, 2021.
(60) Provisional application No. 63/129,313, filed on Dec. 22, 2020.

(51) Int. Cl. A61B 34/30; A61B 34/00

(52) U.S. Cl. CPC A61B 34/30; A61B 34/74; A61B 2017/00477

(57) ABSTRACT

A surgical robotic arm of a surgical robotic system comprising articulation segments that are mechanically and operatively coupled together to form one or more joints. The articulation segments include a rotary actuation mechanism having a male segment assembly having one or more structural components that are rotatable by one or more cables about a longitudinal axis thereof and are rotatable to an extent greater than 360 degrees, and a female segment assembly sized and configured for seating the male segment assembly and being operatively coupled thereto.

### BACKGROUND OF THE INVENTION

The present invention is directed to minimally invasive surgical devices and associated methods, and is more specifically related to robotic surgical systems that are insertable into a patent to perform a selected surgery therein.

Since its inception in the early 1990s, the field of minimally invasive surgery has grown rapidly. While minimally invasive surgery vastly improves patient outcome, this improvement comes at a cost to the surgeon's ability to operate with precision and ease. During laparoscopy, the surgeon must insert laparoscopic instruments through a small incision in the patient's abdominal wall. The nature of tool insertion through the abdominal wall constrains the motion of laparoscopic instruments as laparoscopic instruments cannot move side-to-side without injury to the abdominal wall. Standard laparoscopic instruments are limited to four axes of motion. These four axes of motion are movement of the instrument in and out of the trocar (axis 1), rotation of the instrument within the trocar (axis 2), and angular movement of the trocar in two planes while maintaining the pivot point of the trocar's entry into the abdominal cavity (axes 3 and 4). For over two decades, the majority of minimally invasive surgery has been performed with only these four degrees of motion.

Existing robotic surgical devices attempted to solve many of these problems. Some existing robotic surgical devices replicate non-robotic laparoscopic surgery with additional degrees of freedom at the end of the instrument. However, even with many costly changes to the surgical procedure, existing robotic surgical devices have failed to provide improved patient outcome in the majority of procedures for which they are used. Additionally, existing robotic devices create increased separation between the surgeon and surgical end-effectors. This increased separation causes injuries resulting from the surgeon's misunderstanding of the motion and the force applied by the robotic device. Because the multiple degrees of freedom of many existing robotic devices are unfamiliar to a human operator, such as a surgeon, the surgeons typically undergo extensive training on robotic simulators before operating on a patient in order to minimize the likelihood of causing inadvertent injury to the patient.

To control existing robotic devices, a surgeon sits at a console and controls manipulators with his or her hands and feet. Additionally, robot cameras remain in a semi-fixed location, and are moved by a combined foot and hand motion from the surgeon. These semi-fixed cameras with limited fields of view result in difficulty visualizing the operating field.

Other robotic devices have two robotic manipulators inserted through a single incision. These devices reduce the number of incisions required to a single incision, often in the umbilicus. However, existing single-incision robotic devices have significant shortcomings stemming from their actuator design. Existing single-incision robotic devices include servomotors, encoders, gearboxes, and all other actuation devices within the in vivo robot. This decision to include the motors and gearboxes within the patient's body has resulted in large robots with limited capability. Such a large robot must be inserted through a large incision, thus increasing risk of herniation, risk of infection, pain, and general morbidity. Additionally, it is unlikely that the size of these devices will ever significantly decrease due to the inclusion of motors, gears, etc. within the in vivo devices. This increased incision size results in significantly increased injury to the patient and vastly reduces the practicality of existing devices.

Existing single incision devices also have limited degrees of freedom. Some of these degrees of freedom are non-intuitive to a human, for example elongation of the arm during a procedure. These degrees of freedom require a user interface where the surgeon must make non-intuitive learned movements similar the movements existing multi-incision devices.

In the prior Vicarious Surgical robotic systems, the robot arms have articulation segments or sections and end effectors that can be manipulated and moved in multiple degrees of freedoms using selected mechanical arrangements. Specifically, the robot arms can employ pulleys and associated cables or wires that traverse along an elongated shaft and can connect the various sections of robot arms. A drive assembly that is coupled to the cables can be used to provide actuation forces on the cables to actuate and/or manipulate the arm sections and end-effectors of the instrument. The drive assembly can be mounted on the robot arm or coupled to another portion of the surgical robotic system that is controlled by a surgeon in order to move the robot arms with various degrees of freedoms.

Further, any rotary joint of the robot arms has a limited range of travel determined by the amount of drive cable that can be spooled up, or due to the amount of extra slack in the electrical wires that typically pass through each joint of the robot arm so as to carry signals, such as joint angle measurement, back to the controller and the surgeon. When the rotation of the various joints reaches or exceeds a mechanical limit, there typically is a mechanical failure of the electrical wires and/or drive cables.

### SUMMARY OF THE INVENTION

The present invention is directed to a cable-driven rotary joint of a robot arm of a surgical robotic system that is capable of multiple rotations (e.g., at least two rotations in either direction) with a hard stop formed at each end of the joint travel, while concomitantly having the ability to accurately determine via a series of sensors the number of revolutions or turns of the joint, as well as the specific rotational angle of the joint, upon startup and use of the surgical robotic system. The rotary joint is thus rotatable about a longitudinal axis to an extent greater than 360 degrees.

The present invention is thus directed to a surgical robotic system that employs a robotic arm having an articulation segment that includes a male segment assembly that seats within and is rotatable relative to a female segment assembly. The articulation segment can be located at, and form part of, a joint of the robotic arm, such as a shoulder joint, an elbow joint or a wrist joint. The male segment assembly includes structure that includes a groove, such as a spiral or helical groove, formed on an outer surface. The female segment assembly includes structure that includes a slot that mounts a shuttle assembly that includes a magnet. The shuttle assembly moves linearly or axially within the slot as the male segment assembly is rotated. The position of the shuttle assembly as well as a sensing magnet can be determined by a series of sensors that are mounted relative to the slot and shuttle assembly so as to sense the position of the shuttle magnet and the sensing magnet. The surgical robotic system can then determine, based on the position of the shuttle and sensing magnets, the number of turns of the articulation segment as well as the rotational angle (partial turn position) of the articulation segment.

The present invention is directed to a surgical robotic arm of a surgical robotic system having a plurality of articulation segments that are mechanically and operatively coupled together to form one or more joints. The articulation segment can include a rotary actuation mechanism having a male segment assembly having one or more structural components that are rotatable by one or more cables about a longitudinal axis thereof and are rotatable to an extent greater than 360 degrees, and a female segment assembly sized and configured for seating the male segment assembly and being operatively coupled thereto.

The female segment assembly includes a linear slot and the structural component can include a groove, such as for example a spiral groove, and the robotic arm further includes a shuttle assembly having a shuttle element sized and configured for contacting the groove such that the shuttle element moves linearly in the slot as the male segment component is rotated. The robotic arm can also include, according to one embodiment, a first sensor assembly configured to sense a linear position of the shuttle element in the slot and to generate first sensor data. The first sensor data is processed to determine one or more of the number of turns of the one or more structural components of the male segment assembly and the rotational angular position of the one or more structural components. The robotic arm can include an optional second sensor assembly that is configured to sense a rotational angular position of the structural component of the male segment assembly and for generating second sensor data. The first sensor data and the second sensor data can be processed by a computing unit to determine a rotational position of the joint.

According to another embodiment of the present invention, the surgical robotic arm can include a sensor assembly that is coupled to either the male segment assembly or the female segment assembly for sensing a rotational position of the structural component of the male segment assembly. The sensing assembly can include a first plurality of sensors for generating first sensor data indicative of a number of rotations of the one or more structural components of the male segment assembly, and a second plurality of sensors for generating second sensor data indicative of a rotational angular position of the one or more structural components of the male segment assembly. The first sensor data and the second sensor data can be processed to determine the rotational position of the structural component of the male segment assembly.

The structural component of the male segment assembly can have a groove formed therein for seating a shuttle element configured for traveling along a path. The groove has a first hard stop formed at one end of the path and a second hard stop formed at an opposed end of the path, and the shuttle element is movable within or along the groove upon rotation of the male segment assembly to an extent greater than 360 degrees. The first hard stop and the second hard stop determine a maximum number of rotations of the one or more structural components of the male segment assembly. According to one embodiment, the shuttle element can include a bearing element, and the groove can be a circular groove or a spiral groove.

According to one embodiment, the groove is a spiral groove and the female segment assembly has a slot formed therein that is configured and positioned to communicate with at least a portion of the groove. A shuttle assembly can be provided that seats within the slot and can include the shuttle element, a first magnet coupled to one end of the shuttle element, and a bearing element coupled to an opposed end of the shuttle element for contacting the groove. The shuttle assembly is configured to move linearly within the slot. The robotic arm can include a first plurality of sensors are configured to sense the position of the first magnet within the slot and generate in response the first sensor data. The robotic arm can optionally include a second magnet that is coupled to the structural component of the male segment assembly and is rotatable therewith. The optional second plurality of sensors can be configured to sense a rotational angular position of the second magnet and to generate the second sensor data.

The one or more structural components of the male segment can include a rotary shaft element having a main body having an outer surface, and an engagement member coupled to the outer surface of the rotary shaft member. The engagement member has an outer surface that has the groove formed therein. The female segment assembly can include an outer housing element disposed about at least a portion of the engagement element and has the slot formed therein that exposes at least a portion of the groove. The female segment assembly can further include an optional abutment element for axially separating the engagement element from a bearing element disposed about a portion of the outer surface of the rotary shaft element.

The present invention is also directed to a surgical robotic arm of a surgical robotic system that includes a plurality of articulation segments that are mechanically and operatively coupled together. The articulation segment can include a rotary actuation mechanism that includes a male segment assembly having a rotary shaft element having a main body having an outer surface, and an engagement element that is coupled to the outer surface of the rotary shaft member and has a spiral groove formed therein. The rotary actuation mechanism also includes a female segment assembly that has an outer housing element disposed about at least a portion of the engagement element and has a slot formed therein that exposes at least a portion of the groove. The robotic arm can further include a shuttle assembly mounted within the slot formed in the outer housing element such that a portion of the shuttle assembly seats within the groove of the engagement element. The male segment assembly is disposed within the female segment assembly and is rotatable relative thereto about a longitudinal axis and rotatable to an extent greater than about 360 degrees.

The robotic arm of the present invention an also optionally include one or more of a bearing element disposed about a portion of the outer surface of the rotary shaft element, a flange element formed at one end of the rotary shaft element, and a skirt element formed at one end of the engagement element. The female segment assembly can optionally include an abutment element positioned axially between the skirt element of the engagement element and the bearing element for axially separating the engagement element from the bearing element.

The robotic arm of the present invention can also include a first sensor assembly that is configured to sense a position of the shuttle assembly in the slot and to generate first sensor data. The first sensor data is processed by for example a computing unit to determine the number of turns of the male segment assembly and/or the rotational angular position of the male segment assembly. The surgical robotic arm of the present invention can also include an optional second sensor assembly that is configured to sense a rotational angular position of the male segment assembly and for generating second sensor data. A computing unit can be employed for processing the first sensor data and the second sensor data to determine a rotational position of the joint.

According to another embodiment, the surgical robotic arm can include a sensor assembly that is coupled to the male segment assembly or the female segment assembly for sensing a rotational position of the male segment assembly. The sensing assembly can include a first plurality of sensors for generating first sensor data indicative of a number of rotations of the male segment assembly, and a second plurality of sensors for generating second sensor data indicative of a rotational angular position of the male segment assembly. The first sensor data and the second sensor data can be processed to determine the rotational position of the male segment assembly.

The groove has a first hard stop formed at one end and a second hard stop formed at an opposed end. The shuttle assembly is movable within the groove upon rotation of the male segment assembly to an extent greater than 360 degrees, and the first hard stop and the second hard stop determine a maximum number of rotations of the one or more structural components of the male segment assembly. The shuttle assembly can include a bearing element sized and configured for communicating with the groove, a shuttle element having a first end having a recess formed therein for seating a first portion of the bearing element, and a shuttle magnet coupled to a second opposed end of the shuttle element. A first plurality of sensors can be associated with the slot formed in the outer housing element for sensing a linear position of the shuttle magnet in the slot and for generating first sensor data. The first plurality of sensors can include a first slot sensor associated with one end of the slot and a second slot sensor associated with an opposed end of the slot, The first sensor data is indicative of a number of rotations of the male segment assembly, and can be processed to determine a rotational angular position of the male segment assembly.

The surgical robotic arm can also include an optional annular sensing magnet that is rotationally coupled to the rotary shaft element or the engagement element of the male segment assembly. The arm can include an optional second plurality of sensors that are disposed circumferentially about the annular sensing magnet for sensing a rotational angular position of the annular sensing magnet and for generating second sensor data indicative of a rotational angular position of the male segment assembly.

The present invention is also directed to a method for rotating a joint portion of a surgical robotic arm of a surgical robotic system, the method comprising providing the robotic arm having one or more articulation segments that are mechanically and operatively coupled together to form the joint, wherein the articulation segment includes a male segment assembly having one or more rotatable structural components and a female segment assembly sized and configured for seating the male segment assembly, rotating the structural component of the male segment assembly with one or more cables about a longitudinal axis thereof and wherein the structural component is rotatable to an extent greater than about 360 degrees, and sensing with a sensor assembly coupled to one or more of the male segment assembly and the female segment assembly a rotational position of the structural component of the male segment assembly.

The method can also include generating, with the sensor assembly, first sensor data, and determining from the first sensor data a number of rotations of the structural component of the male segment assembly. Further, the method of the present invention can determine from the first sensor data a rotational angular position of the structural component of the male segment assembly.

The present invention can also include generating with the sensor assembly first sensor data, determining from the first sensor data a number of rotations of the structural component of the male segment assembly, generating with the sensor assembly second sensor data, and determining from the second sensor data a rotational angular position of the structural component of the male segment assembly. The first sensor data and the second sensor data can be processed to determine the rotational position.

The method of the present invention can further include providing a groove in the structural component of the male segment assembly for seating a shuttle element configured for traveling along a path, wherein the groove has a first hard stop formed at one end of the path and a second hard stop formed at an opposed end of the path, and upon rotation of the structural component, moving the shuttle element within the groove to an extent greater than 360 degrees. The first hard stop and the second hard stop can determine a maximum number of rotations of the one or more structural components of the male segment assembly. The method also includes associating a first magnet with the shuttle element, and sensing with the sensor assembly a position of the first magnet within the groove. An optional second magnet can be coupled with the structural member of the male segment assembly, and a rotational angular position of the second magnet can be determined by the sensor assembly.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a schematic illustration of the surgical robotic system of the present invention.

FIGS. 2A and 2B are perspective views of a robot arm of a robotic unit according to the teachings of the present invention.

FIG. 3A is a schematic partial cross-sectional view of an articulation segment (rotary actuator section) of a robotic arm showing for example a male segment assembly according to the teachings of the present invention.

FIG. 3B is a schematic partial cross-sectional view of the articulation segment showing for example a female segment assembly according to the teachings of the present invention.

FIG. 4 is a schematic partial cross-sectional perspective view of the articulation segment of the robotic arm showing the shuttle assembly employed therein according to the teachings of the present invention.

FIG. 5A is a schematic partial cross-sectional perspective view of the articulation segment of the robotic arm showing the shuttle assembly disposed at a first hard stop position according to the teachings of the present invention.

FIG. 5B is a partial cross-sectional view of the articulation segment of the robotic arm showing the shuttle assembly disposed at a first hard stop position according to the teachings of the present invention.

FIG. 6 is a schematic partial cross-sectional perspective view of the articulation segment of the robotic arm showing the shuttle assembly position after one full rotation of the male segment assembly according to the teachings of the present invention.

FIG. 7 is a schematic partial cross-sectional perspective view of the articulation segment of the robotic arm showing the shuttle assembly position after two full rotations of the male segment assembly according to the teachings of the present invention.

FIG. 8 is a schematic partial cross-sectional perspective view of the rotary actuator section of the robotic arm showing the shuttle assembly position after three full rotations of the male segment assembly according to the teachings of the present invention.

FIG. 9 is a schematic partial cross-sectional perspective view of the rotary actuator section of the robotic arm showing the shuttle assembly disposed at a second hard stop position after four full rotations of the male segment assembly according to the teachings of the present invention.

FIG. 10 is a schematic partial cross-sectional view of the rotary actuator section of the robotic arm showing an incremental or sensing magnetic element employed therein for determining a rotational position of the articulation segment according to the teachings of the present invention.

FIG. 11 is schematic partial cross-sectional view of the articulation segment of the robotic arm showing an incremental magnetic element and associated printed circuit board with sensors according to the teachings of the present invention.

FIG. 12 is a perspective view of the printed circuit board and associated sensors employed by the articulation segment of the robotic arm according to the teachings of the present invention.

FIG. 13A is a schematic pictorial partial cross-sectional view of a portion of the articulation segment showing the position of the shuttle assembly within a slot formed in the female segment assembly according to the teachings of the present invention.

FIG. 13B is a schematic pictorial partial top view of a portion of the articulation segment showing the position of the shuttle assembly within the slot formed in the female segment assembly according to the teachings of the present invention.

FIG. 14A is a schematic pictorial partial cross-sectional view of a portion of the articulation segment showing the position of a second embodiment of the shuttle assembly within a slot formed in the female segment assembly showing the use of a single sensor according to the teachings of the present invention.

FIG. 14B is a schematic pictorial partial top view of a portion of the articulation segment showing the position of the second embodiment of the shuttle assembly within the slot formed in the female segment assembly showing the use of a single sensor according to the teachings of the present invention.

FIG. 15 is a partial perspective view of a second embodiment of the multi-turn concept of the present invention.

FIG. 16 illustrates yet another embodiment of the multi-turn concept of the present invention.

FIG. 17 is schematic cross-sectional view showing the path of movement of a ball bearing within a groove as the male segment assembly is rotated according to the teachings of the present invention.

### DETAILED DESCRIPTION

In the following description, numerous specific details are set forth regarding the system and method of the present invention and the environment in which the system and method may operate, in order to provide a thorough understanding of the disclosed subject matter. It will be apparent to one skilled in the art, however, that the disclosed subject matter may be practiced without such specific details, and that certain features, which are well known in the art, are not described in detail in order to avoid complication and enhance clarity of the disclosed subject matter. In addition, it will be understood that any examples provided below are merely illustrative and are not to be construed in a limiting manner, and that it is contemplated by the present inventors that other systems, apparatuses, and/or methods can be employed to implement or complement the teachings of the present invention and are deemed to be within the scope of the present invention.

While the system and method of the present invention can be designed for use with one or more surgical robotic systems employed as part of a virtual reality surgical system, the surgical robotic system of the present invention may be employed in connection with any type of surgical system, including for example robotic surgical systems, straight-stick type surgical systems, and laparoscopic systems. Additionally, the system of the present invention may be used in other non-surgical systems, where a user requires access to a myriad of information, while controlling a device or apparatus.

The surgical robotic system 10 of the present invention employs a robotic subsystem 20 that includes a robotic unit 50 that can be inserted into a patient via a trocar through a single incision point or site. The robotic unit 50 is small enough to be deployed in vivo at the surgical site and is sufficiently maneuverable when inserted to be able to move within the body so as to perform various surgical procedures at multiple different points or sites. The robotic unit 50 includes multiple separate robotic arms that are deployable within the patient along different or separate axes. Further, a surgical camera assembly can also be deployed along a separate axis and form part of the robotic unit 50. Thus, the robotic unit 50 employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be utilized, including but not limited to robotic surgical instruments, as well as other surgical instruments known in the art.

The system and method disclosed herein can be incorporated and utilized with the robotic surgical device and associated system disclosed for example in U.S. Pat. No. 10,285,765 and in PCT patent application Serial No. PCT/US20/39203, and/or with the camera assembly and system disclosed in United States Publication No. 2019/0076199, where the content and teachings of all of the foregoing patents, patent applications and publications are incorporated herein by reference. The robotic unit 50 that forms part of the present invention can form part of the robotic subsystem 20, which in turn forms part of a surgical robotic system 10 that includes a surgeon or user workstation that includes appropriate sensors and displays, and a robot support system (RSS), for interacting with and supporting the robotic unit of the present invention. The robotic subsystem 20 can include, in one embodiment, a portion of the RSS, such as for example a motor assembly and associated mechanical linkages, and the surgical robotic unit 50 can include one or more robot arms and one or more camera assemblies. The surgical robotic unit 50 can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In still other embodiments, the structure may be free standing and portable or movable. The robot support system can mount the motor assembly that is coupled to the surgical robotic unit and can include gears, motors, drivetrains, electronics, and the like, for powering the components of the surgical robotic unit.

The robot arms and the camera assembly are capable of multiple degrees of freedom of movement. According to one practice, when the robot arms and the camera assembly are inserted into a patient through the trocar, they are capable of movement in at least the axial, yaw, pitch, and roll directions. The robot arm assemblies are designed to incorporate and utilize a multi-degree of freedom of movement robotic arm with an end effector region mounted at a distal end thereof that corresponds to a wrist and hand area or joint of the user. In other embodiments, the working end (e.g., the end effector end) of the robot arm is designed to incorporate and utilize other robotic surgical instruments, such as for example the surgical instruments set forth in U.S. Publ. No. 2018/0221102, the contents of which are herein incorporated by reference.

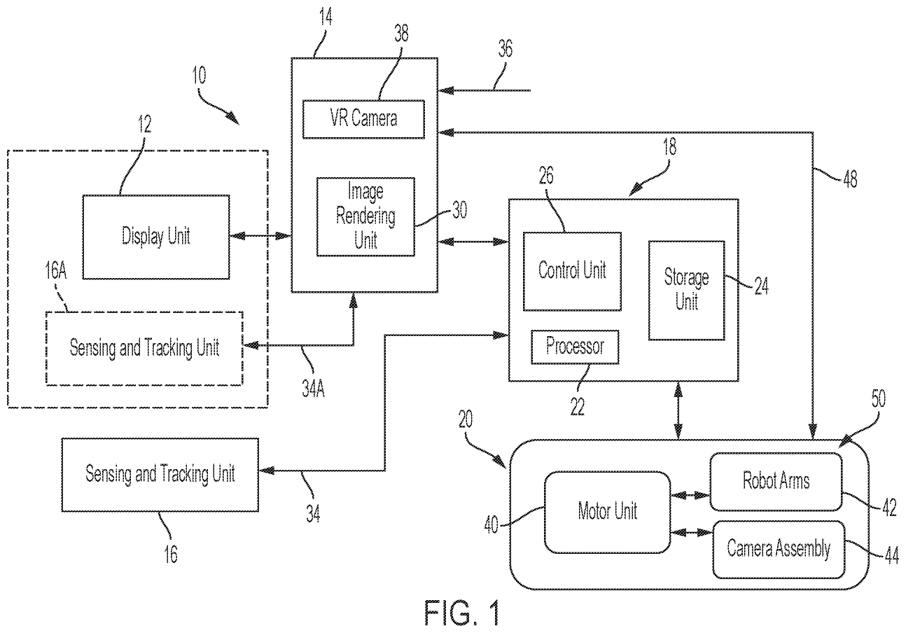

FIG. 1 is a schematic block diagram illustration of a surgical robotic system 10 according to the teachings of the present invention. The system 10 includes a display device or unit 12, a virtual reality (VR) computing unit 14, a sensing and tracking unit 16, a computing unit 18, and a robotic subsystem 20. The display unit 12 can be any selected type of display for displaying information, images or video generated by the VR computing unit 14, the computing unit 18, and/or the robotic subsystem 20. The display unit 12 can include or form part of, for example, a head-mounted display (HMD), a screen or display, a three-dimensional (3D) screen, and the like. The display can form part of the surgeon or user's work station.

The display unit 12 can also include an optional sensor and tracking unit 16A, such as can be found in commercially available head mounted displays. The sensing and tracking units 16 and 16A can include one or more sensors or detectors that are coupled to a user of the system, such as for example a nurse or a surgeon. The sensors can be coupled to the arms of the user and if a head-mounted display is not used, then additional sensors can also be coupled to a head and/or neck region of the user. The sensors in this arrangement are represented by the sensor and tracking unit 16. If the user employs a head-mounted display, then the eyes, head and/or neck sensors and associated tracking technology can be built-in or employed within that device, and hence form part of the optional sensor and tracking unit 16A. The sensors of the sensor and tracking unit 16 that are coupled to the arms of the surgeon can be preferably coupled to selected regions of the arm, such as for example the shoulder region, the elbow region, the wrist or hand region, and if desired the fingers. According to one practice, the sensors from part of a pair of hand controllers that are manipulated by the surgeon. The sensors generate position data indicative of the position of the selected portion of the user. The sensing and tracking units 16 and/or 16A can be utilized to control movement of the camera assembly 44 and the robotic arms 42 of the robotic subsystem 20. The position data 34 generated by the sensors of the sensor and tracking unit 16 can be conveyed to the computing unit 18 for processing by a processor 22. The computing unit 20 can determine or calculate from the position data 34 the position and/or orientation of each portion of the surgeon's arm and convey this data to the robotic subsystem 20. According to an alternate embodiment, the sensing and tracking unit 16 can employ sensors coupled to the torso of the surgeon or any other body part. Further, the sensing and tracking unit 16 can employ in addition to the sensors an Inertial Momentum Unit (IMU) having for example an accelerometer, gyroscope, magnetometer, and a motion processor. The addition of a magnetometer is standard practice in the field as magnetic heading allows for reduction in sensor drift about the vertical axis. Alternate embodiments also include sensors placed in surgical material such as gloves, surgical scrubs, or a surgical gown. The sensors may be reusable or disposable. Further, sensors can be disposed external of the user, such as at fixed locations in a room, such as an operating room. The external sensors can generate external data 36 that can be processed by the computing unit and hence employed by the system 10. In other embodiments, there are sensors located on a mechanical linkage that the user manipulates. The sensors generate signals that serve as inputs to be processed by the computing unit. According to another embodiment, when the display unit 12 is a head mounted device that employs an associated sensor and tracking unit 16A, the device generates tracking and position data 34A that is received and processed by the VR computing unit 14. Further, the sensor and tracking unit 16 include if desired a hand controller. The displays, sensing and tracking units, VR computing unit and the like can form part of a surgeon or remote work station.

In the embodiment where the display is a HMD, the display unit 12 can be a virtual reality head-mounted display, such as for example the Oculus Rift, the Varjo VR-1 or the HTC Vive Pro Eye. The HMD can provide the user with a display that is coupled or mounted to the head of the user, lenses to allow a focused view of the display, and a sensor and/or tracking system 16A to provide position and orientation tracking of the display. The position and orientation sensor system can include for example accelerometers, gyroscopes, magnetometers, motion processors, infrared tracking, eye tracking, computer vision, emission and sensing of alternating magnetic fields, and any other method of tracking at least one of position and orientation, or any combination thereof. As is known, the HMD can provide image data from the camera assembly 44 to the right and left eyes of the surgeon. In order to maintain a virtual reality experience for the surgeon, the sensor system can track the position and orientation of the surgeon's head, and then relay the data to the VR computing unit 14, and if desired to the computing unit 18. The computing unit 18 can further adjust the pan and tilt of the camera assembly 44 of the robot so as to follow the movement of the user's head.

The sensor or position data 34A generated by the sensors if associated with the HMD, such as for example associated with the display unit 12 and/or tracking unit 16A, can be conveyed to the computing unit 18 either directly or via the VR computing unit 14. Likewise, the tracking and position data 34 generated by the other sensors in the system, such as from the sensing and tracking unit 16 that can be associated with the user's arms and hands, can be conveyed to the computing unit 18. The tracking and position data 34, 34A can be processed by the processor 22 and can be stored for example in the storage unit 24. The tracking and position data 34, 34A can also be used by the control unit 26, which in response can generate control signals for controlling movement of one or more portions of the robotic subsystem 20. The surgical robotic system 10 can include a surgeon or user workstation, the robot support system (RSS), and the robotic subsystem 20, and the robotic subsystem 20 can include the motor unit 40 and an implantable robotic unit 50 that includes one or more robot arms 42 and one or more camera assemblies 44. According to another embodiment, the motor unit 40 can form part of the robot support system. The implantable robot arms 42 and the camera assembly 44 can form part of a single support axis robotic unit or subsystem, such as that disclosed and described in U.S. Pat. No. 10,285,765, or can form part of a split arm (SA) architecture robot system, such as that disclosed and described in PCT patent application no. PCT/US20/39203.

The control signals generated by the control unit 26 can be received by the motor unit 40 of the robotic subsystem 20. The motor unit 40 can include a series of servomotors and gears that are configured for driving separately the robot arms 42 and the cameras assembly 44 of the robotic subsystem 50. The robot arms 42 can be controlled to follow the scaled-down movement or motion of the surgeon's arms as sensed by the associated sensors. The robot arms 42 can have portions or regions that can be associated with movements associated with the shoulder, elbow, and wrist joints as well as the fingers of the user. For example, the robotic elbow joint can follow the position and orientation of the human elbow, and the robotic wrist joint can follow the position and orientation of the human wrist. The robot arms 42 can also have associated therewith end regions that can terminate in end-effectors or graspers that follow the movement of one or more fingers of the user, such as for example the index finger as the user pinches together the index finger and thumb. While the arms of the robot follow movement of the arms of the user, the robot shoulders are fixed in position. In one embodiment, the position and orientation of the torso of the user is subtracted from the position and orientation of the users arms. This subtraction allows the user to move his or her torso without the robot arms moving.

The robot camera assembly 44 is configured to provide the surgeon with image data 48, such as for example a live video feed of an operation or surgical site, as well as enable a surgeon to actuate and control the cameras forming part of the camera assembly 44. The camera assembly 44 preferably includes a pair of cameras, the optical axes of which are axially spaced apart by a selected distance, known as the inter-camera distance, so as to provide a stereoscopic view or image of the surgical site. The surgeon can control the movement of the cameras either through movement of a head-mounted display or via sensors coupled to the head of the surgeon, or by using a hand controller or sensors tracking the user's head or arm motions, thus enabling the surgeon to obtain a desired view of an operation site in an intuitive and natural manner. The cameras are movable in multiple directions, including for example in the yaw, pitch and roll directions, as is known. The components of the stereoscopic cameras can be configured to provide a user experience that feels natural and comfortable. In some embodiments, the interaxial distance between the cameras can be modified to adjust the depth of the operation site perceived by the user.

According to one embodiment, the camera assembly 44 can be actuated by movement of the surgeon's head. For example, during an operation, if the surgeon wishes to view an object located above the current field of view (FOV), the surgeon looks in the upward direction, which results in the stereoscopic cameras being rotated upward about a pitch axis from the user's perspective. The image or video data 48 generated by the camera assembly 44 can be displayed on the display unit 12. If the display unit 12 is a head-mounted display, the display can include the built-in tracking and sensor system 16A that obtains raw orientation data for the yaw, pitch and roll directions of the HMD as well as positional data in Cartesian space (x, y, z) of the HMD. However, alternative tracking systems may be used to provide supplementary position and orientation tracking data of the display in lieu of or in addition to the built-in tracking system of the HMD.

The image data 48 generated by the camera assembly 44 can be conveyed to the virtual reality (VR) computing unit 14 and can be processed by the VR or image rendering unit 30. The image data 48 can include still photographs or image data as well as video data. The VR rendering unit 30 can include suitable hardware and software for processing the image data and then rendering the image data for display by the display unit 12, as is known in the art. Further, the VR rendering unit 30 can combine the image data received from the camera assembly 44 with information associated with the position and orientation of the cameras in the camera assembly, as well as information associated with the position and orientation of the head of the surgeon. With this information, the VR rendering unit 30 can generate an output video or image rendering signal and transmit this signal to the display unit 12. That is, the VR rendering unit 30 renders the position and orientation readings of the hand controllers and the head position of the surgeon for display in the display unit, such as for example in a HMD worn by the surgeon.

The VR computing unit 14 can also include a virtual reality (VR) camera unit 38 for generating one or more virtual reality (VR) cameras for use or emplacement in the VR world that is displayed in the display unit 12. The VR camera unit 38 can generate one or more virtual cameras in a virtual world, and which can be employed by the system 10 to render the images for the head-mounted display. This ensures that the VR camera always renders the same views that the user wearing the head-mounted display sees to a cube map. In one embodiment, a single VR camera can be used and in another embodiment separate left and right eye VR cameras can be employed to render onto separate left and right eye cube maps in the display to provide a stereo view. The FOV setting of the VR camera can self-configure itself to the FOV published by the camera assembly 44. In addition to providing a contextual background for the live camera views or image data, the cube map can be used to generate dynamic reflections on virtual objects. This effect allows reflective surfaces on virtual objects to pick up reflections from the cube map, making these objects appear to the user as if they're actually reflecting the real world environment.

The robotic unit 50 can employ multiple different robotic arms 42 that are deployable along different or separate axes. Further, the camera assembly 44, which can employ multiple different camera elements, can also be deployed along a common separate axis. Thus, the robotic unit 50 employs multiple different components, such as a pair of separate robotic arms and a camera assembly 44, which are deployable along different axes. Further, the robot arms 42 and the camera assembly 44 are separately manipulatable, maneuverable, and movable. The robotic subsystem 20, which includes the robot arms and the camera assembly, is disposable along separate manipulatable axes to form the SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through the trocar. By way of example, a surgical instrument can be inserted through the trocar to access and perform an operation in vivo in a body cavity of a patient. In some embodiments, various surgical instruments may be utilized, including but not limited to robotic surgical instruments, as well as other surgical instruments known in the art.

In some embodiments, the robotic subsystem 20 of the present invention is supported by the RSS with multiple degrees of freedom such that the robotic arms 42 and camera assembly 44 can be maneuvered within the patient into a single position or multiple different positions. In one embodiment, the robotic subsystem 20 can be directly mounted to the RSS. In other embodiments, the RSS of the surgical robotic system 10 can optionally include the motor unit 40 that is coupled to the robotic unit 50 at one end and to an adjustable support member or element at an opposed end. Alternatively, as shown herein, the motor unit 40 can form part of the robotic subsystem 20. The motor unit 40 can include gears, one or more motors, drivetrains, electronics, and the like, for powering and driving one or more components of the robot arms and the camera assembly (e.g., robotic unit 50). The robotic unit 50 can be selectively coupled to the motor unit 40. According to one embodiment, the RSS can include a support member that has the motor unit coupled to a distal end thereof. The motor unit 40 in turn can be coupled to the camera assembly 44 and to each of the robot arms 42. The support member can be configured and controlled to move linearly, or in any other selected direction or orientation, one or more components of the robotic unit 50.

The motor unit 40 can also provide mechanical power, electrical power, mechanical communication, and electrical communication to the robotic unit 50, and can further include an optional controller for processing input data from one or more of the system components (e.g., the display 12, the sensing and tracking unit 16, the robot arms 42, the camera assembly 44, and the like), and for generating control signals in response thereto. The motor unit 40 can also include a storage element for storing data. Alternatively, the motor unit 40 can be controlled by the computing unit 18. The motor unit 40 can thus generate signals for controlling one or more motors that in turn can control and drive the robot arms 42, including for example the position and orientation of each articulating joint of each arm, as well as the camera assembly 44. The motor unit 40 can further provide for a translational or linear degree of freedom that is first utilized to insert and remove each component of the robotic unit 50 through a suitable medical device, such as a trocar 108. The motor unit 40 can also be employed to adjust the inserted depth of each robot arm 42 when inserted into the patient 100 through the trocar 108.

The present invention is directed to the ability to swap out tools that form the end effectors of the robot arms of the present invention in an easy and efficient manner. The ability to easily swap out tools allows the user, such as the surgeon, to only remove and replace the end effector portion of the robot arm rather than replace the entire robot arm, which typically has a dedicated tool attached thereto. The tool element removal and replacement can be done within or external to the patient. Since the entire robot arm does not need to be replaced, the robot arm of the present invention reduces costs and waste since the user does not need to employ an entire suite of robot arms and associated tools.

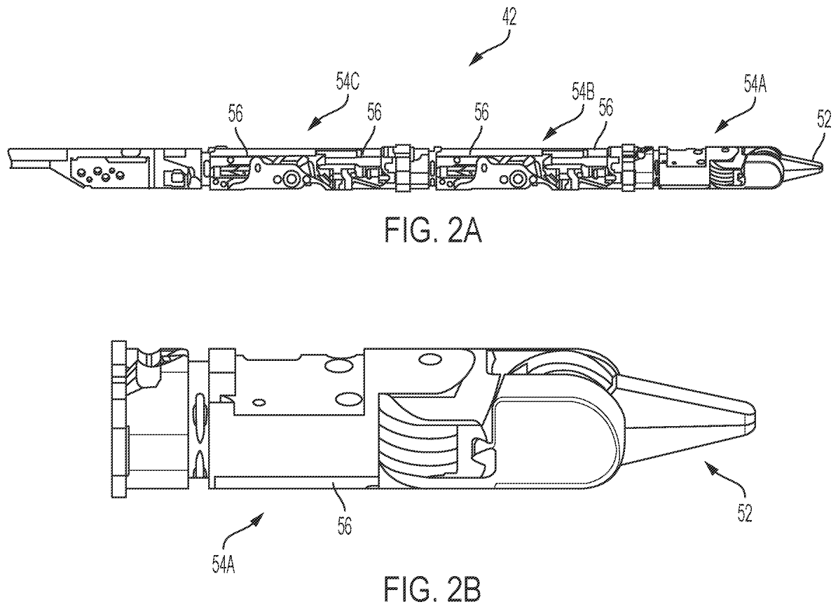

FIGS. 2A and 2B illustrate the general design of selected components of a robot arm 42 of the surgical robotic unit 50 according to the teachings of the present invention. The illustrated robot arm can be cable-driven by suitable cables and employs end effectors 52 at an end effector region or portion of the arm 42. The end effectors provide for a highly functional, easy to use, mechanical connection that allows for the grasping and manipulation of devices or tissue. For the sake of simplicity, only a single robot arm 42 is shown, although a second robot arm or subsequent robot arms can be similar or identical in form and function. The illustrated robot arm 42 can include a series of articulation segments 56 that form joint sections that correspond to the joints of a human arm. For example, the joint section 54A of the robot arm 42 forms the wrist joint, the joint section 54B forms the elbow joint, and the joint section 54C forms the shoulder joint. As such, the articulation segments 56 can be constructed and combined to provide for rotational and/or hinged movement so as to emulate the joints of the human arm. The articulation segments 56 of the robot arm 42 are constructed to provide cable-driven, rotational movement, for example, but within the confines of reasonable rotational limits. The articulation segments 56 are configured to provide maximum torque and speed with minimum size. The articulation segments 56 are mechanically coupled together and end in the end effector portion or segment 52. As shown in FIG. 2B, the end effector portion 52 and the adjacent arm segment 56 form the wrist joint 54A of the robot arm 42.

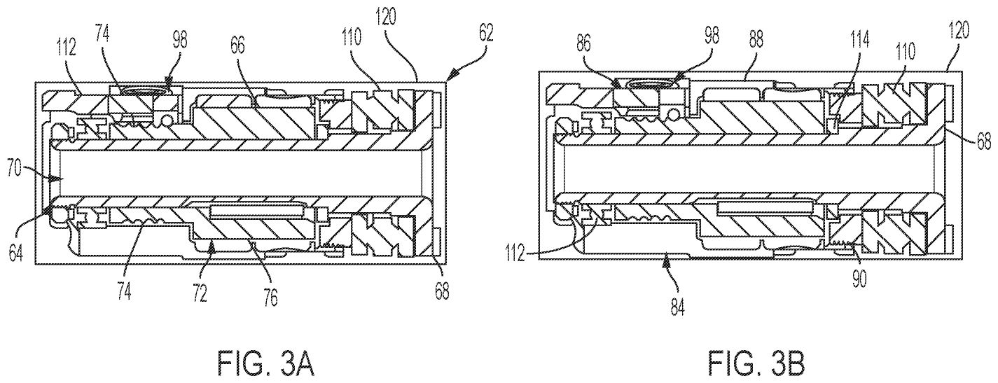

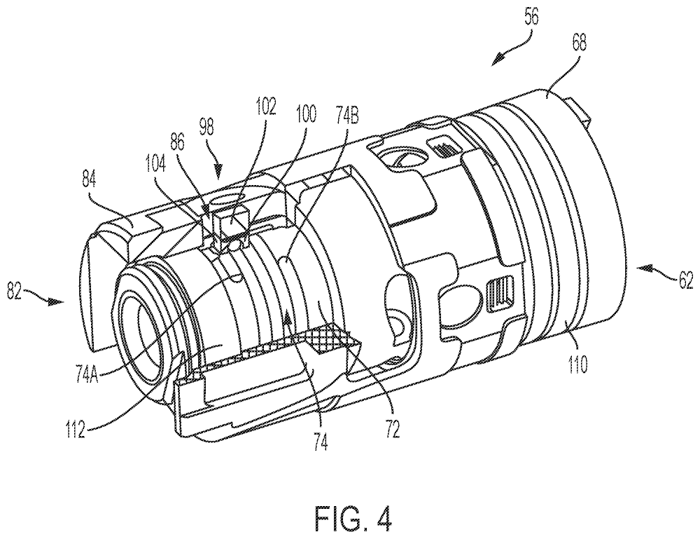

With reference to FIGS. 3A-9, one or more of the articulation segments 56 of the robot arm 42 can be constructed of multiple mechanical components that form a rotary actuation mechanism that can rotate relative to each other in order to effectuate rotational movement of the segments or joints. The articulation segments 56 can be constructed such that the computing unit 18 can easily and readily determine the rotational position of the portions of the articulation segment. The articulation segment 56 includes a main body 60 that includes two main functional groups of components, namely, a male segment assembly 62 and a female segment assembly 82 to form the rotary actuation mechanism. The male segment assembly 62 and the female segment assembly 82 are housed within a main housing 120. The male segment assembly 62 has the freedom to rotate relative to the female assembly 72, similar to the function of a bearing. The male segment assembly is rotated by suitable cables that are coupled thereto, as is known in the art. The illustrated male segment assembly 62 can include a rotary shaft element 64 that has a main body having an outer surface 66. The rotary shaft element 64 also has a flange element 68 formed at one end and has an inner chamber 70. The inner chamber 70 can allow for wires and cables to pass through the articulation segment. The male segment assembly 62 also includes an engagement element 72 that seats on and contacts the outer surface 66 of the rotary shaft element 64 through known mechanical interference and fastening techniques, such brazing, welding, gluing and the like. As such, the engagement element 72 is rotationally coupled to the shaft element 64. The engagement element 72 has an outer surface that includes a groove 74 formed in the outer surface at one end and has an opposed skirt element 76 formed at an opposed end. The groove 74 can be any selected type of groove, and preferably is a helical or spiral shaped groove. In this regard, the groove 74 can be helically formed about a portion of the outer surface of the engagement element 72, and the groove 74 is configured so as to form stop elements 74A, 74B at either end of the groove, FIG. 4. The articulation segment 56 further includes a rear bearing element 110 mounted about the outer surface 66 of the rotary shaft element 64. The bearing element 82 is disposed adjacent to and preferably contacts the flange 68 of the rotary shaft element 64. The articulation segment 56 also includes a front bearing element 112 that is disposed about the outer surface 66 of the rotary shaft member at an end opposite the flange 68. The flange element 112 is disposed adjacent to an axial end portion of the engagement element 72. The bearing elements 110, 112 help the male segment assembly 62 easily rotate when mounted within the female segment assembly 82 and the main housing 120. The male segment assembly can be rotated by one or more cables.

The illustrated female segment assembly 82 is sized and dimensioned for accommodating the male segment assembly 62. The female segment assembly 82 can include an outer housing element 84 that seats about the outer surface of the engagement element 72. The outer housing element 84 can include a tail portion 88 that is disposed at an end portion that overlies the skirt element 76 of the engagement element 72. The outer housing element 84 forms the outer portion of the female segment assembly 82 and can include a space or slot 86 formed in the outer surface for seating a shuttle assembly 98. The shuttle assembly 98 is configured to move linearly or axially within the slot 86 when the male segment assembly 62 is rotated. The shuttle assembly can be any component or series of components that are configured to communicate with or contact a portion of the groove so as to travel therealong. According to one embodiment, the shuttle assembly 98 can include a shuttle element 100 that has a magnet 102 coupled thereto at one end. The shuttle element 100 also includes and seats at an opposed end a groove contacting element, such as a bearing 104, that is sized and configured for seating within the groove 74 and within the shuttle element 100. According to an alternate embodiment, the shuttle assembly 98 simply includes the shuttle element, without the need for the bearing or the magnet, for contacting the groove 74 and for moving or riding along the groove as the male segment assembly 62 is rotated. The shuttle element can have any selected shape, size, or configuration suitable for directly or indirectly communicating with the groove 74 and/or the slot 86. The rotational movement of the male segment assembly 62 translates rotational movement thereof to linear or longitudinal movement of the shuttle assembly 98, as the bearing 104 tracks or moves along the groove 74 when the male segment assembly 62 is rotated. The stop elements 74A, 74B formed along the endpoints of the groove 74 function as groove end indicators so as to define the range of movement of the bearing within the groove 74 and hence the range of axial movement of the shuttle assembly 98 within the slot 86. The stop elements also serve as end points that define the maximum number of rotations of the male segment assembly. The female segment assembly 82 also includes an abutment element 90 that is axially disposed between the bearing element 110 and the outermost end face of the skirt element 76 of the engagement element 72. The abutment element 90 functions as a mechanical separator for separating the bearing element 110 from the rotatable engagement element 72. The outer housing element 84 that can be coupled to one or more proximal components of the robotic arm 42, and the rotary shaft element 64 of the male assembly 62 can be attached to one or more distal components of the robotic arm 42.

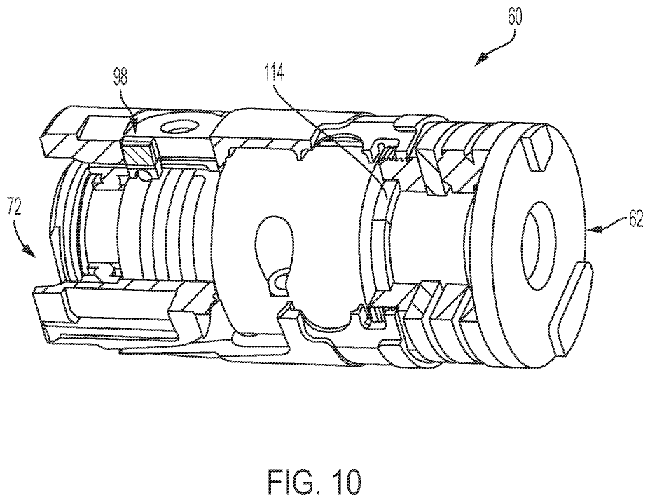

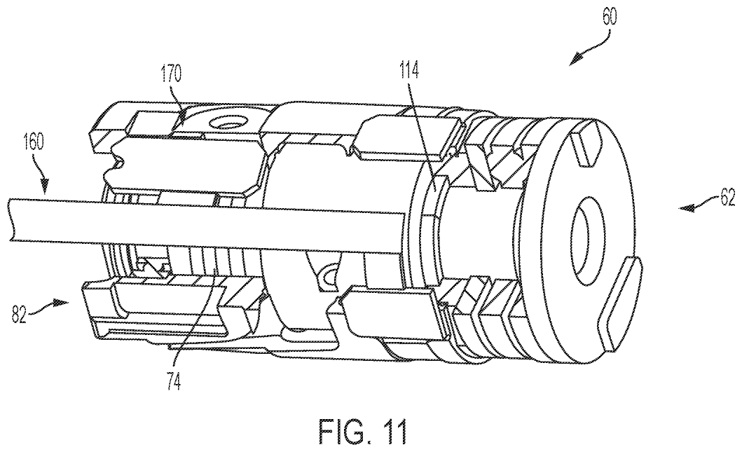

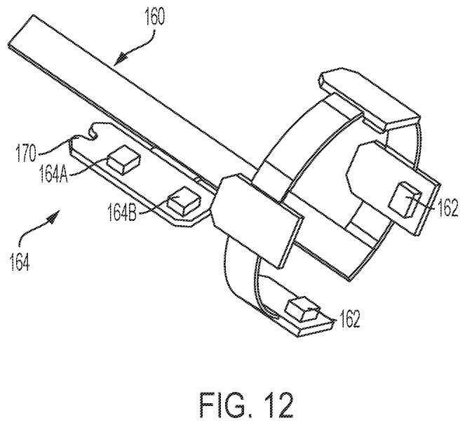

Referring to FIGS. 3A-3B and 10-12, the articulation segment 56 can further include other structure, such as, for example, a sensing magnet 114 that is coupled to the rotary shaft element 64 of the male assembly 62 and rotates therewith. The sensing magnet 114 is employed when the system senses and determines the incremental rotational angle of the male segment assembly 62. Specifically, the male segment assembly 62 can be rotated a selected number of turns, where each turn corresponds to a full and complete 360 degrees. The articulation segment 56 can also include a printed circuit board (PCB) 160 that is coupled to the outer housing element 84 of the female assembly 82 for mounting and positioning a series of sensor elements, including sensors and magnets, for measuring the relative rotational movement or motion in the joint formed by the articulation segment 56. For example, the PCB 160 can employ a series of position sensors 162 for generating sensor data indicative of the relative rotational position of the sensing magnet 114 that rotates with the rotary shaft element 64. The sensors 162 can be any selected type of sensor, and can include Hall effect sensors. When the PCB 160 is mounted to the female segment assembly 82, the sensors 162 are generally circumferentially aligned with the annular sensing magnet 114 so as to be able to sense the rotational position of the magnet 114. By simple way of example, the sensing magnet 114 rotates with the rotary shaft element 64 and the sensors 162 positioned about the magnet 114 sense the rotational or angular position of the magnet. The sensors 162 generate sensor data in response to the position of the magnet 114 that is received by the computing unit 18, which in turn determines a specific rotational angle or position of the magnet and hence of the male segment assembly 62 (e.g., partial turn position). Hence, if the male segment assembly 62 and the associated sensing magnet 114 is rotated a quarter turn, then the sensors 162 sense the in-turn or partial turn rotational angular position of the magnet 114, and the computing unit 18 correlates the sensed electromagnetic strength of the sensing magnet 114 to a specific rotational angle, which in the current example is 90 degrees.

The PCB 160 can also include additional sensors 164, such as Hall Effect sensors, that are mounted on a section 170 of the PCB 160. The PCB section 170 can be mounted adjacent to the slot 86 and the sensors 164A and 164B mounted thereon can be positioned so as to be at opposed ends of the slot 86, and hence at opposite ends of the groove 74. The shuttle element 100 and associated magnet 102 interact with the sensors 164A, 164B to measure the relative position of the magnet 102 between the sensors 164A, 164B. The magnet 102 moves along a linear path within the slot 86 between the additional sensors 164A, 164B and is driven by the relative motion between the male assembly 62 and the female assembly 82. The linear travel of the shuttle assembly 98 is proportional to the full multi-turn range of motion (e.g., +/−two rotations from a neutral position) of the rotary shaft member 64 and associated engagement element 72. Additionally, those of ordinary skill in the art will readily recognize that the linear travel of the shuttle assembly can correspond to any selected number of total rotations of the male segment assembly, and preferably include greater than one full rotation. The slot 86 allows the shuttle assembly 98 to move back and forth in the linear slot 86 since the bearing 104 travels within and along the groove 74 when the engagement member is rotated. Hence, the relative motion of the surfaces of the male segment assembly 62 and the female segment assembly 82 rotates the groove 74 in a selected direction and linearly moves the shuttle assembly 98 within the slot, similar to a ball-screw mechanism. The sensors 164A, 164B sense the relative position of the magnet 102 of the shuttle assembly 98 in the slot 86 as the shuttle assembly approaches either hard stop 74A, 74B, and this information is processed by the computing unit 18 to determine the absolute rotational position as well as the number of full rotational turns of the rotary shaft element 64 of the male assembly 62. Specifically, the position of the shuttle assembly 98 within the slot 86 can be correlated to the position of the bearing 104 within the groove 74, which in turn can be correlated to the rotary position or number of rotations of the male segment assembly 62. According to another embodiment, just the linear position of the shuttle element 100 can be used to calculate the rotational position of the rotary shaft member 64 within its full range of motion (>360 degrees). According to still another embodiment, the accuracy of the measurement or determination of the rotary position can be improved by including rotational position data received from the sensing magnet 114 and its associated sensors 162. In this embodiment, the calculated rotational position and associated signals from the sensors 162 is periodic within one full rotation of the joint and thus are unable to sense the rotational position of the male assembly beyond one full turn. Thus, rotating the rotary shaft element 64 and hence the joint more than one full rotation may cause the signal to ‘wrap’ or reset and the absolute rotational position is unknown. This is overcome by incorporating the sensor data generated by the slot sensors 164A, 164B, which can be utilized by the computing unit 18 to determine the number of full rotations of the male segment assembly or components thereof. The computing unit 18 can process the sensor data from the sensors 164 that is indicative of the number of full rotations of the male segment assembly 62 as well as the sensor data from the sensors 162 that is indicative of the partial rotation of the male segment assembly and generate based thereon an absolute rotational position of the male segment assembly. The sensor data can be correlated to the number of rotational turns by comparing the measured signal strength from each sensor to prestored measurements or by using simple algorithms and device geometry. The articulation segment 56 can employ an incremental sensing assembly that includes the sensing magnet 114 and associated sensors 162 that in combination are capable of providing relatively high resolution joint rotational angle information to the computing unit 18 within a single rotation, but typically does not determine the precise number of rotational turns and associated turn count of the joint. As such, the incremental sensors (e.g., sensors 114 and 162) can assist in determining the relative rotational angle of the joint of the robotic arm but not the specific number of rotations of the robotic arm. This information combined with the sensing data provided by the additional sensors 164 enables the system to determine the specific number of rotations of the male segment assembly 62, and hence of the joint. Those of ordinary skill in the art will readily recognize that the sensor data generated by the sensors 164 can also be employed to determine the rotational angle of the male segment assembly, and as such the sensors 162 and the sensing magnet 114 can be optional components of the surgical robotic arm.

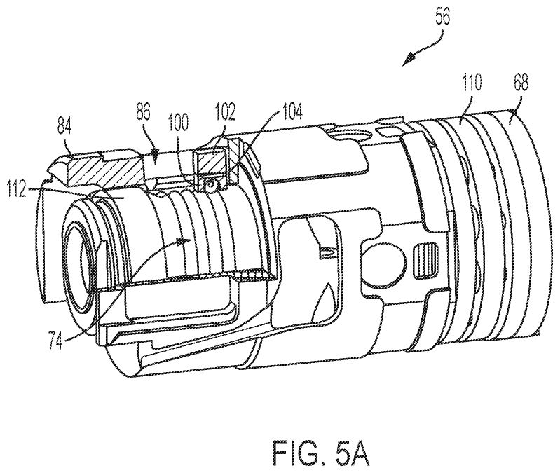

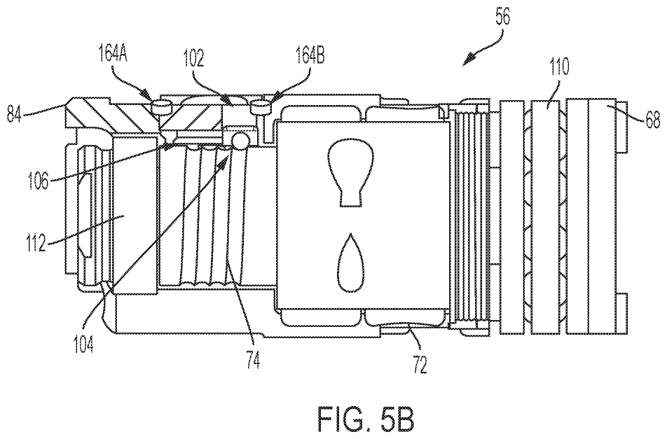

FIGS. 4-10 and 13A-13B illustrate the relative movement of the bearing 104 in the spiral or helical groove 74. Specifically, as the male segment assembly 62 is rotated relative to the female segment assembly 82, the bearing 104 moves along the spiral groove 74 in a direction counter or opposite to the direction of rotation of the male segment assembly. The bearing 104 is coupled to the shuttle element 100, and hence the rotational movement of the rotary shaft element 64 and associated engagement member 66, along with movement of the bearing 104 in the spiral groove 74 converts or translates the rotational movement of the male segment assembly (e.g., the rotary shaft element 64 and associated engagement element 72) into linear movement of the shuttle assembly 98. As such, the shuttle assembly 98 moves linearly or axially back and forth in the slot 86 along a linear path 106. The male segment assembly 62 can be rotated a sufficient number of turns such that the bearing 104 reaches the hard stop 74B formed in the groove 74, as shown in FIGS. 5A and 5B. In this position, the shuttle element 100 and the associated magnet 102 are positioned immediately adjacent to the sensor 164B and to the sensing magnet 114. In this position, the sensor 164B senses the strongest magnetic force and associated polarity from the magnet 102, and the sensor 164B generates sensor data indicative of the position of the magnet and hence of the shuttle assembly. The sensor data can be processed such that the system 10 can determine from the sensor data that the joint has been rotated to a maximum rotary position and amount of turns in a selected direction. The male segment assembly 62 can then be rotated in the opposite direction to move the ball bearing 104 away from the hard stop 74B, and correspondingly move the shuttle element 100 and the magnet 102 away from the sensor 164B and towards the opposite sensor 164A. The system can store preselected information such that the system can determine that the maximum sensor signals from either sensor 164A and 164B is indicative of or corresponds to a rotary position that corresponds to about two full turns from a neutral or center position (e.g., +/−2 full turns). The signal from the sensor 164B with the bearing 104 located at the stop 74B is indicative of the articulation segment 56 having been rotated at a maximum extent. According to one embodiment, the male segment assembly is rotated two full turns from the center or neutral position. The sensor data generated by the sensors 164A, 164B cannot be utilized by the computing unit 18 to determine the rotational angle of the male segment assembly 62. As such, the system 10 can employ the sensors 162 and the sensing magnet 114 to determine the rotational position of the male segment assembly 62 within a single turn (e.g., partial turn angle).

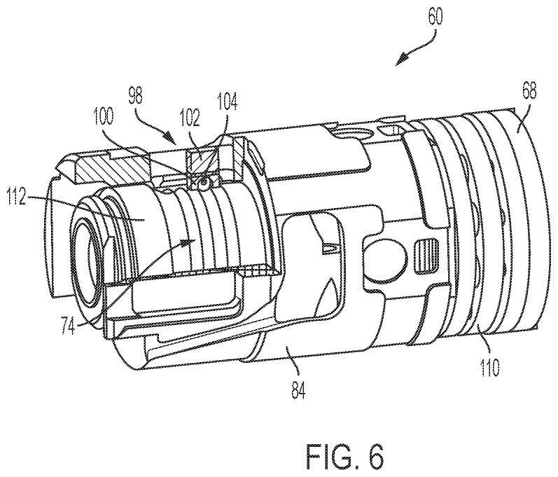

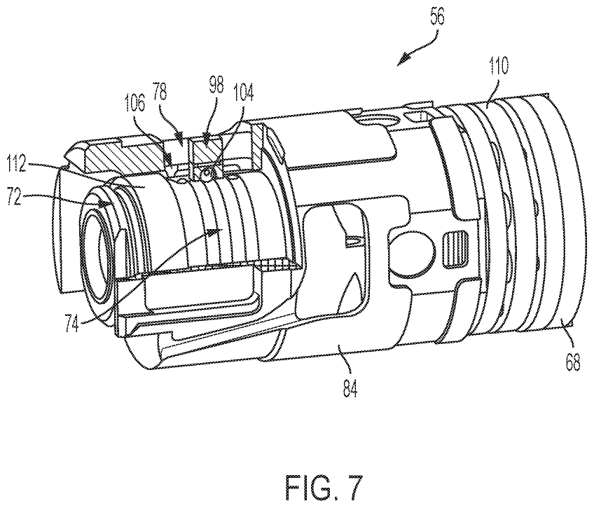

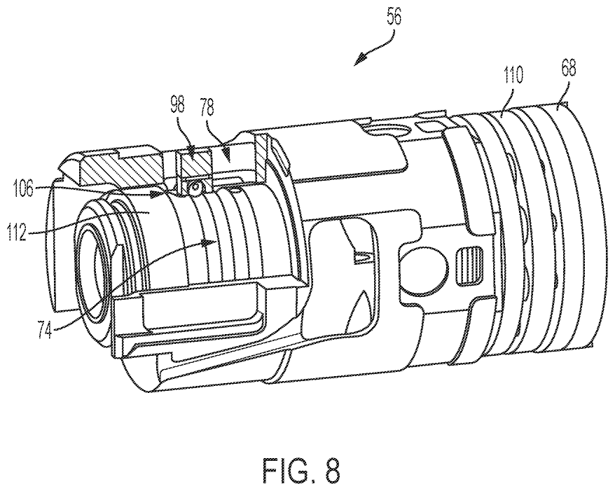

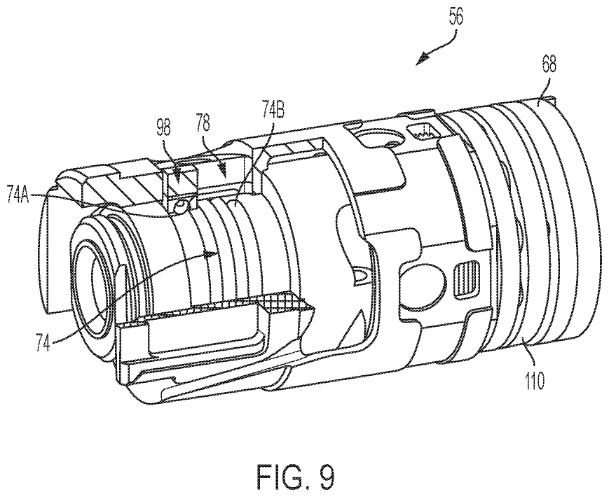

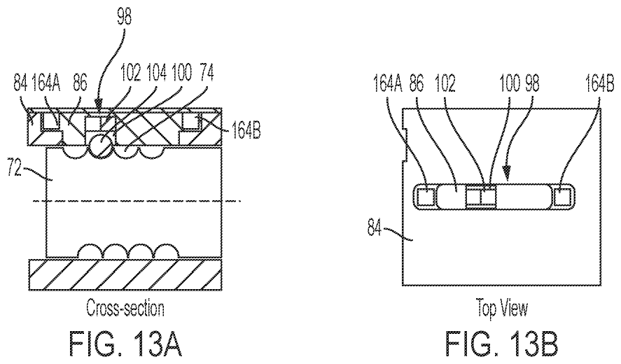

FIG. 6 illustrates the position of the shuttle assembly 98 after one full rotation of the male assembly 62 in the opposite direction. In this position, the bearing 104 is positioned in the second row of the spiral groove 74. The shuttle assembly 98 can be moved from this position in either direction along the slot 86 depending upon the rotational direction of the male segment assembly 62. FIG. 7 illustrates the position of bearing 104 in the third row of the spiral groove 74 after two full rotations of the male segment assembly 62. The illustrated position can correspond to or can be considered a net neutral or center rotational position since it is situated at a midpoint between the stops 74A and 74B. The shuttle assembly 98 is thus moved linearly along the linear path 106 further away from the sensor 164B and closer to the sensor 164A. FIGS. 8, 13A and 13B show the position of the bearing 104 after three full rotations of the male segment assembly 62. After four full rotations of the male segment assembly 62, the bearing 104 reaches or contacts the opposed hard stop 74A, as shown in FIG. 9. In this position, the shuttle element 100 and the magnet 102 are positioned immediately adjacent to the sensor 164A. The sensor data generated by the sensors 164A, 164B, and which is indicative of the relative linear position of the magnet 102, can be correlated by the computing unit 18 to the specific number of turns or rotations of the male segment assembly 62. For example, when the sensor 164A generates a signal having a maximum strength or magnitude, then the system 10 can determine from the signal magnitude that the articulation segment 56 has been rotated to a maximum extent in that particular direction. The signal strength or magnitude from the sensors 164A, 164B can be correlated to the position of the ball bearing 104 and hence to the number of rotations or turns of the male segment assembly 62.

According to another embodiment the present invention, the number of turns or rotations of the male segment assembly 62 as calculated by the computing unit 18 can be used to directly determine the total absolute rotational angle of the male assembly, where the absolute rotational angle is referred to as being one of the two hard stop positions, and is equal to about 900 degrees from a respective hard-stop position. Still further, the sensors 162 and the incremental or sensing magnet 114 can be used to determine the incremental rotational angular position of the male segment assembly 62 within a single rotational turn with greater accuracy. Thus, the magnet 102 and the slot sensors 164A, 164B can determine the absolute number of turns or rotations of the male segment assembly 62, and the incremental sensing magnet 114 and associated sensors 162 can determine the specific incremental rotational angle or position of the assembly 62 within a single rotational turn. In other similar embodiments, the data from the sensors 162 and the incremental magnet element 114, as well as the data from the magnet 102 and the sensors 164A and 164B, can be combined by utilizing a filtering algorithm, such as a Kalman filter or other known filtering algorithms, as is known in the art. The combination of the two sensor data sources has the advantage of significantly increasing the accuracy of the estimate or determination of the rotational angular position of the rotary joint. Overall, this highly accurate rotational position determining methodology of the present invention allows the system 10 to determine at any point, and especially at startup, the position of the rotary joint.

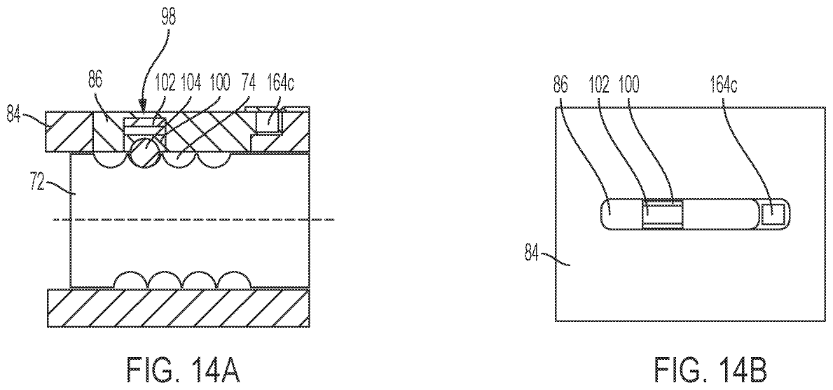

FIGS. 14A and 14B illustrate another embodiment of the articulation segment 56 according to the teachings of the present invention. As shown, the articulation segment 56 can employ just a single sensor 164C associated with the slot 86. The data generated by the sensor 164C, and which is indicative of the relative linear position or location of the magnet 102, can be correlated by the computing unit 18 to the specific number of turns or rotations of the male segment assembly 62. The potential disadvantage to using just a single sensor 164C versus using opposed dual sensors 164A and 164B is a decrease in overall accuracy, but the information generated by the single sensor 164C is suitable for use.

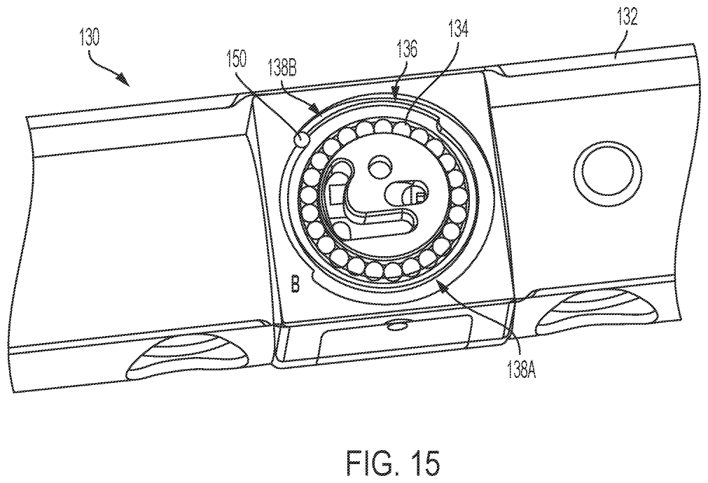

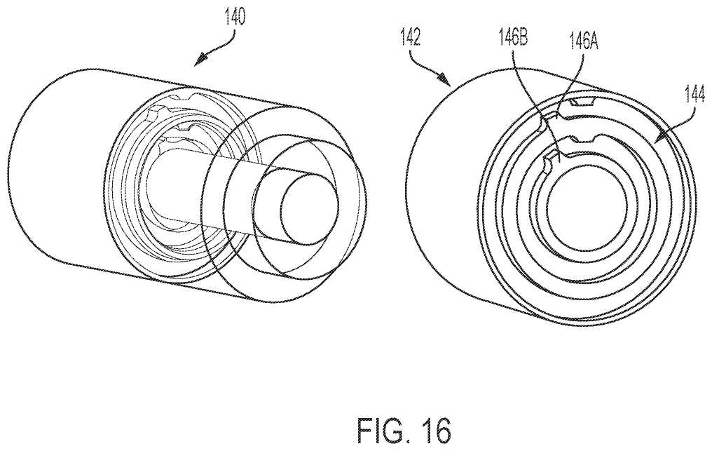

FIG. 15 illustrates another embodiment of the multi-turn rotary actuation mechanism of the present invention. The illustrated rotary actuation mechanism 130 can be employed by the articulation segment 56 of the present invention for implementing multi-turn rotation of, for example, one or more components of the male segment assembly 62, while concomitantly employing or incorporating hard stops in a groove formed therein. The illustrated actuation mechanism 130 includes a housing 132 that includes a plurality of bearings 134 housed therein. The housing 132 includes an engagement mechanism 136 for engaging, for example, the rotary shaft member 64 or the engagement element 72 of the male segment assembly 62. The illustrated rotary actuation mechanism 130 can incorporate, for example, any selected number of rotations by adding stages as depicted in FIG. 16, and preferably allows the components to rotate over a range of 540 degrees. The bearings 134 of the actuator mechanism 130 allow the male segment assembly to rotate relative to the main body 132. The engagement mechanism 136 can include an inner profile 138A and an outer profile 138B having different radii. The hard stop B designates a maximum extent or degree that the rotary actuation mechanism can be rotated.

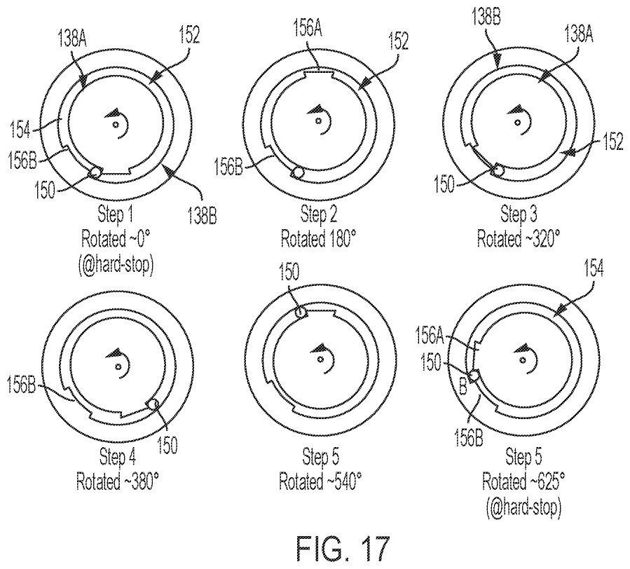

A simplified embodiment of the hard-stop design of the rotary actuation mechanism 130 is illustrated in FIG. 17. FIG. 17 depicts the inner profile 138A and the outer profile 138B in which a single ball bearing 150 is placed and allowed to freely move within or along a circular groove 152 that forms a circular path 154. The inner profile 138A includes an inner protrusion 156A and the outer profile 138B includes an outer protrusion 156B. The opposed sides of the outer protrusion 156B form first and second hard stop elements including the hard stop B, and the inner protrusion 156A when rotated engages with and moves the bearing 150 along the circular path 154. In combination, the inner and outer profiles 138A, 138B constitute one stage of the rotary actuation mechanism 130. The theoretical maximum range of 720 degrees of the current mechanism is typically not achieved due to the mechanical constraints and the overlap required to accommodate the ball bearing 150. The hard stop limits of greater than 720 degrees can be achieved by adding additional stages that can be disposed radially inward or axially in series, if necessary. In the illustrated embodiment, there is a 40 degree section of the outer profile 138B formed by the outer protrusion 156B that is offset and extends radially inwardly, and there is a 40 degree section of the inner profile 138A formed by the inner protrusion 156A that is offset and extends radially outwardly. The angle of the offset section can be arbitrarily selected. As such, about 40 degrees was selected and is appropriate for the total range of circular or rotary motion. The inner profile 138A rotates relative to the outer profile 138B around the bearing axis (i.e., the axis normal to the ring of bearing balls). When the inner profile 138A rotates counter-clockwise about 320 degrees (step 3), the inner protrusion 156A contacts or engages with the bearing 150 and starts to move the bearing along the circular path 154 in a counter-clockwise direction, and the inner and outer 40 degree sections formed by the protrusions 156A, 156B are aligned. When the inner profile 138A continues to rotate counter-clockwise, the protrusion 156A continues to push the ball bearing 150 counter-clockwise in the groove 152 along the circular path 154. When the inner profile 138A and bearing 150 rotate another 305 degrees, the bearing 150 contacts the outer protrusion 156B at the second or other hard stop at point “B,” as shown in step 5. As such, the total range of motion between the two hard stops formed by the outer protrusion 156B can be calculated as follows:

2N−N*(θi+θo+2θb) Equ. 1

where N equals the number of stages, Oi equals the angle of the inner section, θo equals the angle of the outer section, and θb equals the angle that the bearing 150 takes up on the profiles.

FIG. 16 is yet another embodiment of the multi-turn rotary actuation mechanism of the present invention. The present invention includes a rotary assembly 140 that can be incorporated into the articulation segment 56, and includes a rotary element 142 that has a series of concentric rotational grooves or stages 144 formed therein. The grooves 144 of the rotary element 142 can include one or more hard stop elements 146A, 146B. The grooves 144 mirror in some respects the inner and outer profiles 138A, 138B, respectively, of the actuation mechanism 130. In order to expand the motion range to four full rotations the inner and outer profiles 138A, 138B are repeated by the concentric nature of the grooves 144. This results in a hard stop at either end of the rotational range. Additional stages can be added to increase the range of the hard stops.

It will thus be seen that the invention efficiently attains the objects set forth above, among those made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

Having described the invention, what is claimed as new and desired to be secured by Letters Patent is:

1. A surgical robotic arm of a surgical robotic system, comprising a plurality of articulation segments that are mechanically and operatively coupled together to form one or more joints, wherein the one or more of the plurality of articulation segments includes a rotary actuation mechanism having
a male segment assembly having one or more structural components that are rotatable by one or more cables about a longitudinal axis thereof and are rotatable to an extent greater than 360 degrees, and
a female segment assembly sized and configured for seating the male segment assembly and being operatively coupled thereto.

2. The surgical robotic arm of claim 1, wherein the female segment assembly includes a linear slot and wherein the one or more structural components includes a groove, further comprising a shuttle assembly having a shuttle element sized and configured for contacting the groove such that the shuttle element moves linearly in the slot as the male segment component is rotated.

3. The surgical robotic arm of claim 2, further comprising a first sensor assembly configured to sense a linear position of the shuttle element in the slot and to generate first sensor data.

4. The surgical robotic arm of claim 3, wherein the first sensor data is processed to determine one or more of the number of turns of the one or more structural components of the male segment assembly and the rotational angular position of the one or more structural components.

5. The surgical robotic arm of claim 3, further comprising a second sensor assembly configured to sense a rotational angle of the one or more structural components of the male segment assembly and for generating second sensor data.

6. The surgical robotic arm of claim 5, further comprising a computing unit for processing the first sensor data and the second sensor data to determine a rotational position of the joint.

7. The surgical robotic arm of claim 1, further comprising a sensor assembly coupled to one or more of the male segment assembly and the female segment assembly for sensing a rotational position of the one or more structural components of the male segment assembly.

8. The surgical robotic arm of claim 7, wherein the sensing assembly comprises
a first plurality of sensors for generating first sensor data indicative of a number of rotations of the one or more structural components of the male segment assembly, and
a second plurality of sensors for generating second sensor data indicative of a rotational angular position of the one or more structural components of the male segment assembly, and wherein the first sensor data and the second sensor data can be processed to determine the rotational position of the one or more structural components of the male segment assembly.

9. The surgical robotic arm of claim 8, wherein the one or more structural components of the male segment assembly includes a groove formed therein for seating a shuttle element configured for traveling along a path, wherein the groove has a first hard stop formed at one end of the path and a second hard stop formed at an opposed end of the path, and wherein the shuttle element is movable within the groove upon rotation of the male segment assembly to an extent greater than 360 degrees, and wherein the first hard stop and the second hard stop determine a maximum number of rotations of the one or more structural components of the male segment assembly.

10. The surgical robotic arm of claim 9, wherein the shuttle element is a bearing.

11. The surgical robotic arm of claim 9, wherein the groove is a circular groove or a spiral groove.

12. The surgical robotic arm of claim 9, wherein the groove is a spiral groove, and wherein the female segment assembly includes a slot formed therein that is configured and positioned to communicate with at least a portion of the groove.

13. The surgical robotic arm of claim 12, further comprising a shuttle assembly having the shuttle element, a first magnet coupled to one end of the shuttle element, and a bearing coupled to an opposed end of the shuttle element for contacting the groove, and wherein the shuttle assembly is configured to move linearly within the slot.

14. The surgical robotic arm of claim 13, wherein the first plurality of sensors are configured to sense the position of the first magnet within the slot and to generate the first sensor data.

15. The surgical robotic arm of claim 14, further comprising a second magnet coupled to the one or more structural components of the male segment assembly and rotatable therewith, wherein the second plurality of sensors are configured to sense a rotational angular position of the second magnet and generate the second sensor data.

16. The surgical robotic arm of claim 8, wherein the surgical robotic system includes a computing unit configured for receiving
the first sensor data and for determining, based on the first sensor data, the number of rotations of the one or more structural components of the male segment assembly, and
the second sensor data and for determining, based on the second sensor data, the rotational angular position of the one or more structural components of the male segment assembly.

17. The surgical robotic arm of claim 15, wherein the one or more structural components of the male segment assembly comprises
a rotary shaft element having a main body having an outer surface, and
an engagement member coupled to the outer surface of the rotary shaft member, wherein the engagement member has an outer surface having the groove formed therein.

18. The surgical robotic arm of claim 17, wherein the female segment assembly comprises an outer housing element disposed about at least a portion of the engagement element and having the slot formed therein that exposes at least a portion of the groove.

19. The surgical robotic arm of claim 18, wherein the female segment assembly further comprises an abutment element for axially separating the engagement element from a bearing element disposed about a portion of the outer surface of the rotary shaft element.

20. A surgical robotic arm of a surgical robotic system, comprising a plurality of articulation segments that are mechanically and operatively coupled together, wherein one or more of the plurality of articulation segments includes a rotary actuation mechanism that includes
a male segment assembly having
a rotary shaft element having a main body having an outer surface, and
an engagement element coupled to the outer surface of the rotary shaft member and having a spiral groove formed in an outer surface thereof,
a female segment assembly having an outer housing element disposed about at least a portion of the engagement element and having a slot formed therein that exposes at least a portion of the groove, and
a shuttle assembly mounted within the slot formed in the outer housing element, wherein a portion of the shuttle assembly seats within the groove of the engagement element,
wherein the male segment assembly is disposed within the female segment assembly and is rotatable relative thereto about a longitudinal axis and rotatable to an extent greater than about 360 degrees.

21-37. (canceled)

38. A method for rotating a joint portion of a surgical robotic arm of a surgical robotic system, the method comprising
providing the robotic arm having one or more articulation segments that are mechanically and operatively coupled together to form the joint, wherein the articulation segment includes a male segment assembly having one or more rotatable structural components and a female segment assembly sized and configured for seating the male segment assembly,
rotating the structural component of the male segment assembly with one or more cables about a longitudinal axis thereof and wherein the structural component is rotatable to an extent greater than about 360 degrees, and
sensing with a sensor assembly coupled to one or more of the male segment assembly and the female segment assembly a rotational position of the structural component of the male segment assembly.

39. The method of claim 38, further comprising
generating, with the sensor assembly, first sensor data, and
determining from the first sensor data a number of rotations of the structural component of the male segment assembly.

40. The method of claim 39, further comprising determining from the first sensor data a rotational angular position of the structural component of the male segment assembly.

41. The method of claim 38, further comprising
generating with the sensor assembly first sensor data,
determining from the first sensor data a number of rotations of the structural component of the male segment assembly,
generating with the sensor assembly second sensor data, and
determining from the second sensor data a rotational angular position of the structural component of the male segment assembly,
wherein the first sensor data and the second sensor data can be processed to determine the rotational position.

42. The method of claim 41, further comprising
providing a groove in the structural component of the male segment assembly for seating a shuttle element configured for traveling along a path, wherein the groove has a first hard stop formed at one end of the path and a second hard stop formed at an opposed end of the path, and
upon rotation of the structural component, moving the shuttle element within the groove to an extent greater than 360 degrees,
wherein the first hard stop and the second hard stop determine a maximum number of rotations of the one or more structural components of the male segment assembly.

43. The method of claim 42, further comprising
associating a first magnet with the shuttle element, and
sensing with the sensor assembly a position of the first magnet within the groove.

44. The method of claim 43, further comprising
coupling a second magnet with the structural member of the male segment assembly, and
sensing with the sensor assembly a rotational angular position of the second magnet.
